(19) United States
(12) Patent Application Publication
(10) Pub. No.: US 2023/0265485 A1
(43) Pub. Date: Aug. 24, 2023

(54) NUCLEIC ACID ANALYSIS DEVICE, AND NUCLEIC ACID ANALYSIS METHOD

(71) Applicant: CANON KABUSHIKI KAISHA, Tokyo (JP)

(72) Inventor: Sota TORII, Tokyo (JP)

(21) Appl. No.: 18/168,601

(22) Filed: Feb. 14, 2023

(30) Foreign Application Priority Data
Feb. 18, 2022 (JP) 2022-023910

(51) Int. Cl. C12Q 1/6806; G01N 21/64; B01L 3/00

(52) U.S. Cl. CPC C12Q 1/6806; G01N 21/6428; B01L 3/502715; B01L 3/502784; B01L 2300/02; B01L 2200/16; B01L 2300/087

(57) ABSTRACT

Provided is a nucleic acid analysis device with which artifact components are reduced. The nucleic acid analysis device includes a distribution unit, a labeling unit, an information acquisition unit, and an information processing unit. The information processing unit includes an information extraction unit, an artifact component determination unit, a correction unit, and an identifying unit. The artifact component determination unit determines an artifact component from extracted information corresponding to a portion other than a plurality of individual separated compartments. The correction unit uses the artifact component to correct extracted information corresponding to the plurality of individual separated compartments.

### BACKGROUND OF THE INVENTION

### Field of the Invention

The present invention relates to a nucleic acid analysis device, and a nucleic acid analysis method.

### Description of the Related Art

Jennifer Doudna et al. of the University of California have shown that different strains of human papilloma virus (HPV) in a human sample can be accurately detected in distinction from each other through use of Cas12a (Science 27 Apr. 2018: Vol. 360, Issue 6387, pp. 436-439). A complex composed of Cas12a and crRNA specifically recognizes a sequence of target DNA and is bound thereto, and Cas12a cleaves the bound target DNA. In that case, when a reporter molecule in which a fluorescent substance and a quencher are linked to each other by single-stranded DNA is added to a reaction system, Cas12a cleaves the single-stranded DNA of the reporter molecule by a trans-cleavage reaction. Thus, the fluorescent substance and the quencher are separated, and fluorescence is generated. That is, when the target DNA is present in the sample, fluorescence is generated from the fluorescent substance derived from the reporter molecule through activation of the trans-cleavage reaction of Cas12a, and hence the target DNA can be detected based on the fluorescence.

However, as described in Multiplexed single molecule immunoassays, Lab Chip, 2013, 13, 2902, it is known that, when a reaction reagent is isolated in a minute region to acquire a fluorescent image by a fluorescence microscope or the like, signals (artifacts) other than a fluorescence signal occur in the fluorescent image due to various factors. Examples of particularly problematic artifacts include one (shading) ascribable to brightness unevenness of incident light and an influence of light from (optical crosstalk with) an adjacent well. Those artifacts may cause significant deterioration in accuracy of fluorescence detection, and are desired to be removed or reduced.

There have been proposed several methods of reducing artifacts. In regard to the shading, it is disclosed in Japanese Patent Application Laid-Open No. 2003-262588 that an artifact component (shading component) ascribable to the brightness unevenness of the incident light is acquired in advance through use of a diffusion plate or the like and a photographed image is corrected based on the acquired artifact component. In regard to the optical crosstalk, it is disclosed in Multiplexed single molecule immunoassays, Lab Chip, 2013, 13, 2902 that optical crosstalk with an adjacent well is calculated for each pattern and a photographed image is corrected based on the calculated optical crosstalk. However, it is a complicated processing step to acquire a shading component in advance for the purpose of correcting the shading. In addition, the shading component may differ between photographed images, and in that case, effective correction is difficult. Meanwhile, for the optical crosstalk, a processing step of calculating an artifact component (optical crosstalk component) ascribable to an influence of light from an adjacent well is complicated, and it is practically required to consider the optical crosstalk component ascribable to the influence of light from every well. Thus, it is difficult to completely eliminate optical crosstalk.

### SUMMARY OF THE INVENTION

Thus, the present invention has an object to provide a nucleic acid analysis device with which artifacts are reduced without complicating processing steps.

According to one embodiment of the present invention, there is provided a nucleic acid analysis device including: a distribution unit; a labeling unit; an information acquisition unit; and an information processing unit, wherein the information processing unit includes: an information extraction unit; an artifact component determination unit; a correction unit; and an identifying unit, wherein the distribution unit is configured to distribute a sample including nucleic acid and a reagent to a plurality of individual separated compartments to form a set including the plurality of individual separated compartments and a portion other than the plurality of individual separated compartments, wherein the labeling unit is configured to cause a change in each of the plurality of individual separated compartments to which the sample including target nucleic acid is distributed so that the each of the plurality of individual separated compartments to which the sample including target nucleic acid is distributed and each of the plurality of individual separated compartments to which a sample excluding target nucleic acid is distributed are distinguishable from each other by the identifying unit, wherein the information acquisition unit is configured to acquire information corresponding to the set, wherein the information extraction unit is configured to extract, from the acquired information corresponding to the set, each of information corresponding to the plurality of individual separated compartments and information corresponding to the portion other than the plurality of individual separated compartments, wherein the artifact component determination unit is configured to determine an artifact component from the extracted information corresponding to the portion other than the plurality of individual separated compartments, wherein the correction unit is configured to use the artifact component to correct the extracted information corresponding to the plurality of individual separated compartments, and wherein the identifying unit is configured to identify, based on the corrected information corresponding to the plurality of individual separated compartments, each of the plurality of individual separated compartments in which a magnitude of the change exceeds a predetermined threshold.

Further, according to one embodiment of the present invention, there is provided a nucleic acid analysis method including: a distribution step; a labeling step; an information acquisition step; an information extraction step; an artifact component determination step; a correction step; and an identifying step, wherein the distribution step includes distributing a sample including nucleic acid and a reagent to a plurality of individual separated compartments to form a set including the plurality of individual separated compartments and a portion other than the plurality of individual separated compartments, wherein the labeling step includes causing a change in each of the plurality of individual separated compartments to which the sample including target nucleic acid is distributed so that the each of the plurality of individual separated compartments to which the sample including target nucleic acid is distributed and each of the plurality of individual separated compartments to which a sample excluding target nucleic acid is distributed are distinguishable from each other in the identifying step, wherein the information acquisition step includes acquiring information corresponding to the set, wherein the information extraction step includes extracting, from the acquired information corresponding to the set, each of information corresponding to the plurality of individual separated compartments and information corresponding to the portion other than the plurality of individual separated compartments, wherein the artifact component determination step includes determining an artifact component from the extracted information corresponding to the portion other than the plurality of individual separated compartments, wherein the correction step includes using the artifact component to correct the extracted information corresponding to the plurality of individual separated compartments, and wherein the identifying step includes identifying, based on the corrected information corresponding to the plurality of individual separated compartments, each of the plurality of individual separated compartments in which a magnitude of the change exceeds a predetermined threshold.

Further, according to one embodiment of the present invention, there is provided a non-transitory storage medium storing a program for causing a computer to execute the nucleic acid analysis method.

Further features of the present invention will become apparent from the following description of exemplary embodiments with reference to the attached drawings.

### BRIEF DESCRIPTION OF THE DRAWINGS

FIG. 1 is a configuration diagram of a nucleic acid analysis device.

FIG. 2 is a cross-sectional view of a well plate.

FIG. 3 is a flow chart for illustrating a flow of a nucleic acid analysis method.

FIG. 4 is a flow chart for correcting an artifact component through estimation.

FIG. 5 is a conceptual diagram of a fluorescent image of the well plate.

FIG. 6 is a flow chart for correcting an artifact component by calculation.

FIG. 7A shows an artifact component estimation image.

FIG. 7B shows an image before correction.

FIG. 7C shows an image after correction.

FIG. 7D shows a profile before correction.

FIG. 7E shows a profile after correction.

### DESCRIPTION OF THE EMBODIMENTS

An embodiment of the present invention is now described in detail with reference to the drawings. However, components described in the embodiment are merely an example. The technical scope of the present invention is defined by the appended claims, and is not limited by the embodiment to be described below.

### Embodiment of the Present Invention

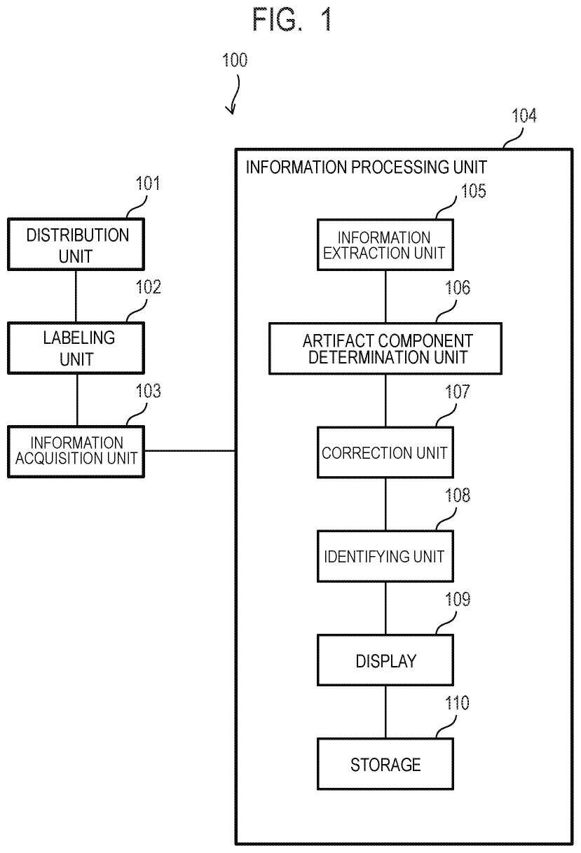

FIG. 1 is a functional block diagram of a nucleic acid analysis device 100 according to the embodiment of the present invention. The nucleic acid analysis device 100 includes a distribution unit 101, a labeling unit 102, an information acquisition unit 103, and an information processing unit 104.

The distribution unit 101 distributes a sample including nucleic acid and a reagent to a plurality of individual separated compartments to form a set including the plurality of individual separated compartments and a portion other than the plurality of individual separated compartments.

The plurality of individual separated compartments can be wells including the sample including nucleic acid and the reagent that have been distributed, and the set can be a well plate including the wells. In this case, the portion other than the plurality of individual separated compartments includes a part of a region other than the wells of the well plate.

In another case, the plurality of individual separated compartments can be liquid droplets including the sample including nucleic acid and the reagent that have been distributed, and the set can include the liquid droplets and a dispersion medium. In this case, the portion other than the plurality of individual separated compartments can include a part of the dispersion medium.

A preferred example of the volume of each individual separated compartment is 0.1 fL or more and 1,000 fL or less, and a further preferred example thereof is 0.5 fL or more and 400 fL or less.

It suffices that the sample includes nucleic acid, and the nucleic acid includes DNA and RNA. A substance derived from a living body, an extract from a living body or the like, blood, a blood-derived substance, food, a food-derived substance, a natural product, a natural product-derived substance, or a culture-medium-derived substance can be used as the sample. The reagent can contain, for example, an effector protein, crRNA to be bound to target nucleic acid, and a reporter molecule.

The labeling unit 102 causes a change in each individual separated compartment to which the sample including target nucleic acid is distributed so that each individual separated compartment to which the sample including target nucleic acid is distributed and each individual separated compartment to which a sample excluding target nucleic acid is distributed are distinguishable from each other by an identifying unit 108. Generation of fluorescence can be used as the change, and in that case, information corresponding to the set, which is acquired by the information acquisition unit 103, includes a fluorescent image of the set. For example, the labeling unit 102 activates the effector protein through binding of crRNA to the target nucleic acid, and modifies the reporter molecule by the activated effector protein, to thereby be able to generate fluorescence. The target nucleic acid can be nucleic acid having any sequence, and can be, for example, nucleic acid that can be applied to diagnosis of a disease state, constitution diagnosis, or the like. Examples of the disease state include a cancer, an autoimmune disease, and an infectious disease, and examples of the infectious disease include infectious diseases caused by DNA viruses, RNA viruses, and the like.

The information acquisition unit 103 acquires information corresponding to the set. For example, the information acquisition unit 103 can detect fluorescence as such information.

The information processing unit 104 identifies an individual separated compartment in which a magnitude of the change exceeds a predetermined threshold based on the information corresponding to the set.

The information processing unit 104 includes an information extraction unit 105, an artifact component determination unit 106, a correction unit 107, and the identifying unit 108. Those components are described later.

The information processing unit 104 may include a display 109, and the display 109 displays a result of calculation performed by the information processing unit 104 on a monitor or the like. The information processing unit 104 may also include a storage 110. The information processing unit 104 can have functions of a computer. For example, the information processing unit 104 may be configured integrally with a desktop personal computer (PC), a laptop PC, a tablet PC, a smartphone, or the like. The information processing unit 104 may further have functions of controlling operations of the distribution unit 101, the labeling unit 102, and the information acquisition unit 103 in accordance with a predetermined program. In order to implement functions as a computer that performs computation and storage, the information processing unit 104 can include a central processing unit (CPU), a random access memory (RAM), a read only memory (ROM), and a hard disk drive (HDD). The information processing unit 104 may also include a communication interface (I/F), a display device, and an input device.

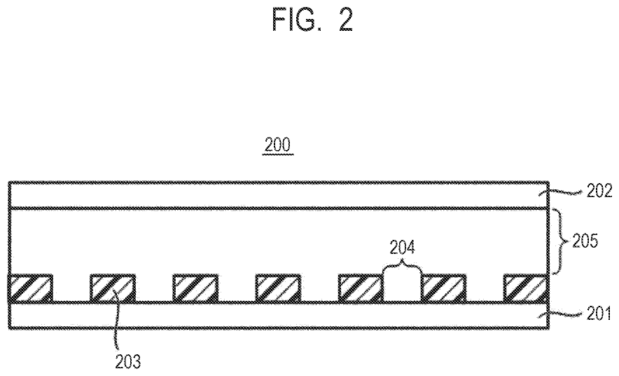

The nucleic acid analysis device according to the embodiment of the present invention can be used for nucleic acid analysis using a CRISPR-Cas technology. In that case, when a sample including target nucleic acid is distributed to the individual independent separated compartments, the sample is apparently brought into a concentrated state, and it is possible to detect the target nucleic acid without performing an amplification step and shorten a time period for fluorescence signal saturation. In addition, when the volume per compartment of the individual independent separated compartments is sufficiently reduced, the target nucleic acid included in one compartment can be set to one molecule or less, and when the number of compartments from which fluorescence signals are obtained is counted, it is possible to calculate a concentration of the target nucleic acid in the sample. In this case, the plurality of individual separated compartments can be liquid droplets including the sample including nucleic acid and the reagent that have been distributed. A preferred example of the liquid droplet is a water-in-oil type emulsion (W/O emulsion). In another case, the plurality of individual separated compartments can be wells including the sample including nucleic acid and the reagent that have been distributed. As an example of the wells, it is possible to use wells included in a well plate having such a structure as illustrated in FIG. 2. In FIG. 2, there are illustrated a well plate 200, a lower substrate 201, an upper substrate 202, partition walls 203, wells 204, and a space 205.

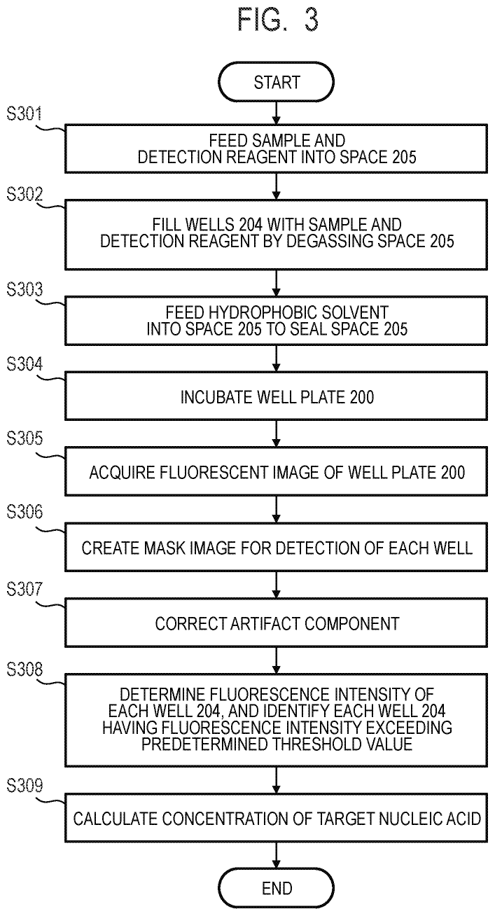

FIG. 3 is a flow chart for illustrating a flow of the nucleic acid analysis in the embodiment of the present invention. A specific example of the nucleic acid analysis is described in detail with reference to FIG. 3.

Step S301 and Step S302 are executed by the distribution unit 101. A reaction liquid composed of a sample and a detection reagent is fed from an injection port portion (not shown) into the space 205 of the well plate 200 in which the injection port portion and a discharge port portion (not shown) are opened.

In Step S302, the wells 204 are filled with the reaction liquid. As a reaction liquid filling method, for example, there is a method of leaving the well plate 200 under reduced pressure and degassing the space 205. Specifically, it is preferred to leave the well plate 200 in a vacuum desiccator of 0.1 atm for a predetermined time period. Through degassing, the air in the wells 204 is removed, to thereby be able to efficiently fill the wells 204 with the reaction liquid. A degassing time period is not particularly limited, and can be freely set. The reaction liquid filling method is not limited to a method based on the degassing.

Step S303 and Step S304 are executed by the labeling unit 102. In Step S303, a hydrophobic solvent is fed into the space 205, and the space 205 is sealed. That is, the reaction liquid present in the space 205 above the wells 204 is replaced with the hydrophobic solvent. Examples to be used as the hydrophobic solvent can include a fluorine-based oil, a saturated aliphatic hydrocarbon, an unsaturated aliphatic hydrocarbon, an aromatic hydrocarbon, and a silicone oil. Examples of the fluorine-based oil can include Fluorinert, AsahiKlin AE-3000 (manufactured by AGC Inc.), and Fomblin (manufactured by Solvay S.A.). Examples of the saturated aliphatic hydrocarbon can include Isopar (manufactured by Exxon Mobil Corporation) and a mineral oil. In Step S304, the labeling unit 102 and a fluorescence generator incubate the well plate 200 filled with the reaction liquid in an incubator at 37° C. However, the reaction temperature can be freely set, and is not limited to 37° C. Through this incubation, a trans-cleavage reaction of CRISPR-Cas progresses, and fluorescence derived from a fluorescent substance possessed by the reporter molecule is generated.

Step S305 is executed by the information acquisition unit 103. In Step S305, the incubation set in advance is finished, and a bright-field image and a fluorescent image of each well 204 are acquired. The information acquisition unit 103 may be any device that acquires the bright-field image and the fluorescent image, and a fluorescence microscope is used in this case. The bright-field image is acquired for the purpose of detection of each well. However, when a reporter or the like is added to the reaction liquid so that every well emits light, a fluorescent image acquired under a photographing condition that matches a fluorescence wavelength of the reporter may also be used. In this case, as the reporter to be added to the reaction liquid, it is preferred to select a reporter having a fluorescence wavelength sufficiently distant from the fluorescence wavelength of the reporter to be cleaved by CRISPR-Cas and caused to fluoresce.

Step S306 is executed by the information extraction unit 105. In Step S306, the information extraction unit 105 creates a mask image (well: 1, other than well: 0) of each well through use of the bright-field image. That is, a default mask is created based on a shape of each well, and the created default mask is sent to the storage 110 and shared by the correction unit 107 and the like. As a method of creating the mask image, the following methods are conceivable. For example, the wells are regularly arranged, and hence a method of creating a well mask template in advance and adjusting the translation direction and the angle by template matching or the like is conceivable. In addition, a method of emphasizing the outer periphery of the well through edge detection using a Sobel filter or the like and performing circle detection through a Hough transform or the like is conceivable. Further, the detection may be performed through use of deep learning.

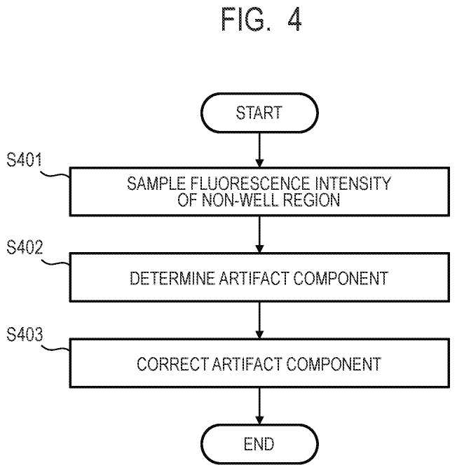

Step S307 is executed by the artifact component determination unit 106 and the correction unit 107 to correct an artifact component of the fluorescent image. A specific example of the flow of correcting the artifact component is described in detail with reference to FIG. 4.

[Determination of Artifact Component]

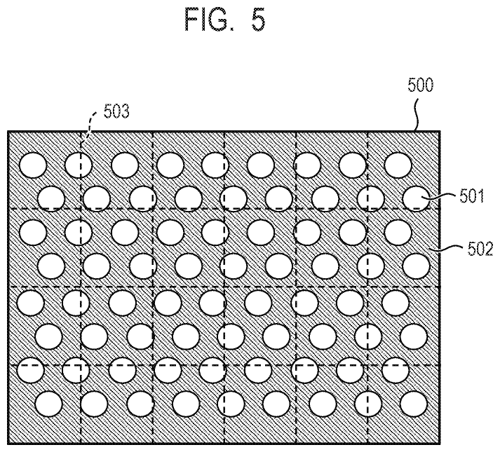

In Step S401, a fluorescence intensity of a non-well region 502 is sampled from the mask image created in Step S306 and a fluorescent image 500 illustrated in FIG. 5 together with coordinate information. As a sampling method, it suffices that the brightness of the fluorescent image at coordinates having a pixel value of 0 in the mask image is sampled.

In Step S402, the correction unit 107 can determine an artifact component through estimation by a method such as two-dimensional curved surface approximation through use of the brightness and the coordinate information sampled in Step S401. The determination of an artifact component through estimation increases accuracy of analysis. As the estimation, for example, basis function approximation, such as radial basis function approximation, or polynomial approximation can be used. In order to avoid overestimating or underestimating an artifact component in a well region, it is preferred to use a linear function as the basis function. In addition, a shading component and an optical crosstalk component are low-frequency components that spread spatially to some extent. In consideration thereof, it is preferred to insert a regularization term at a time of approximation and estimate a smooth curved surface. In another case, a curved surface may be approximated without regularization, and an artifact component that is the approximated curved surface may be blurred by being subjected to a low-pass filter such as a Gaussian filter. The curved surface approximation has a problem in that a calculation cost and a memory usage amount increase as the number of sampling points increases. In particular, a memory amount is often subjected to constraints, and hence it is also possible to reduce the number of sampling points in order to reduce the memory usage amount. In order to achieve accurate curved surface approximation even when the number of sampling points is reduced, the coordinates for sampling are preferred to be dispersed over the entire image. For example, it is possible to uniformly sample the entire fluorescence image 500 by dividing the fluorescent image 500 into small regions of interest (ROIs) 503 delimited by the dotted lines as illustrated in FIG. 5 and sampling the non-well region 502 at a plurality of points in each small ROI.

In Step S403, the correction unit 107 corrects the artifact component by subtracting the artifact component determined in Step S402 from the fluorescent image or dividing the fluorescent image by the artifact component.

In Step S308, the identifier 108 calculates the fluorescence intensity of each well through use of the fluorescent image with the artifact component having been corrected in Step S307, and identifies each well 204 having a fluorescence intensity exceeding a predetermined threshold value (positive determination). The threshold value may be automatically determined from the fluorescence intensities of the respective wells by Otsu's method or the like, or a well plate having a DNA concentration of 0 may be measured in advance and the threshold value may be then determined from variations in fluorescence intensity thereof.

In Step S309, a concentration of the target nucleic acid is calculated from the number of wells generating fluorescence after the trans-cleavage reaction in the incubation of Step S304. When the amount of target nucleic acid included in the sample is large, one liquid droplet may include two or more target nucleic acid molecules. Thus, the number of target nucleic acid molecules may fail to match the number of wells generating fluorescence. For the above-mentioned reason, it is preferred to calculate the concentration of the target nucleic acid through calculation that takes the Poisson distribution into consideration. In the Poisson distribution, when the average number of molecules per liquid droplet is set as λ, a ratio P(k) of the liquid droplets generating fluorescence can be expressed by Expression 1.

P(k)=(λ^{k}/k!)e^{-λ}(k=0,1,2, . . . ) Expression 1

From the number of liquid droplets generating fluorescence, P(k) can be determined and λ, can be calculated. Thus, through use of Expression 1, the concentration of the target nucleic acid can be calculated from the number of liquid droplets in which fluorescence has been detected among all the liquid droplets.

With this embodiment, it is possible to appropriately remove the artifact component generated in the fluorescent image, correct the fluorescence intensity, and improve the fluorescence detection accuracy. This embodiment has been described through use of the well plate, but the individual independent separated compartments may be liquid droplets. When the wells are replaced with liquid droplets, an artifact component can be corrected by the same method as in this embodiment, and analysis accuracy can be improved.

[Determination of Artifact Component through Calculation]

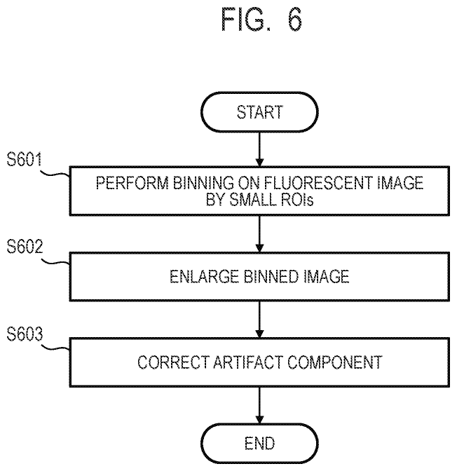

The case in which an artifact component is determined through estimation has been described above. Meanwhile, when an artifact component is determined through estimation, there has been a problem in that increasing the number of points for sampling brightness information leads to a great increase in the number of computation steps and a great increase in memory. When an artifact component is determined through calculation, it is possible to suppress the increase in memory, the increase in the number of computation steps, and an increase in memory consumption. This embodiment is described in detail with reference to FIG. 6. The steps for determining an artifact component are the same except that the estimation of the artifact component is changed to the calculation.

In Step S601, the artifact component determination unit 106 creates a binned image from the mask image created in Step S306 and the fluorescent image 500 illustrated in FIG. 5. As a binning method, it is assumed that each small ROI 503 is replaced with a statistical value such as an average value, and pixel information on a mask region is excluded at a time of averaging. That is, for example, when the fluorescent image is divided into 64×64 small ROIs 503, a binned image having a size of 64×64 is created. The binned image created in this manner is an image that reflects artifact components other than a fluorescence signal component which include a background component, a shading component, and optical crosstalk.

In Step S602, the binned image created in Step S601 is enlarged so as to have the same image size as that of the fluorescent image. That is, an image of the portion other than the individual separated compartments is set as the 64×64 binned image, and this 64×64 binned image is used as the artifact component. For enlargement processing, it is possible to use a bilinear method, a bicubic method, a Lanczos method, or the like. In addition, the shading component and the optical crosstalk component are low-frequency components that spread spatially to some extent. In consideration thereof, the Lanczos method or the bicubic method using information on surroundings is preferred. As another example, an artifact component may be blurred by being subjected to a low-pass filter such as a Gaussian filter so as to avoid overestimating or underestimating the artifact component due to an abrupt change. The enlarged image is called “artifact component image.”

In Step S603, the artifact component is corrected by subtracting the artifact component image created in Step S602 from the fluorescent image or dividing the fluorescent image by the artifact component image.

After the artifact component correction, the process of negative or positive determination and concentration calculation based on the fluorescent image in which the artifact component has been corrected is the same as in the above-mentioned embodiment.

In this manner, through creation of a regular image such as a binned image, it is possible to create an artifact component image by interpolation processing at a relatively low calculation cost.

In order to appropriately correct optical crosstalk that is relatively abrupt compared to shading, it is preferred to determine an artifact component through estimation. However, in order to save memory and computation cost due to hardware constraints, it is preferred to determine an artifact component through calculation.

### Example

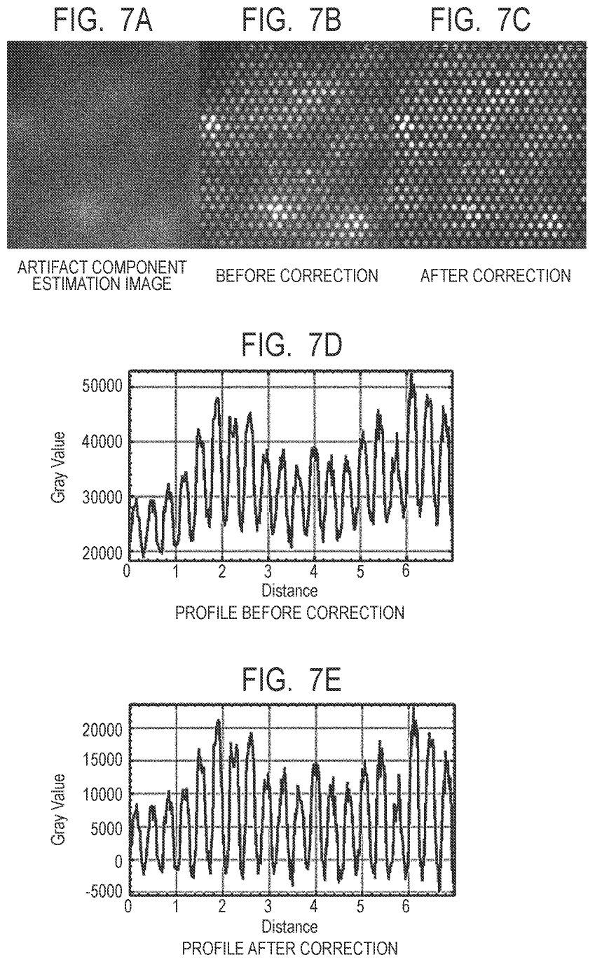

An example in which an artifact component was determined through estimation is described with reference to FIG. 7A to FIG. 7E. FIG. 7B is a fluorescent image before correction of an artifact component. FIG. 7A shows an artifact component estimated from FIG. 7B in this Example. FIG. 7C is a fluorescent image obtained after artifact correction by subtracting FIG. 7A from FIG. 7B. FIG. 7D and FIG. 7E show profiles taken along the upper dotted-line portion in the fluorescent images before and after correction. In FIG. 7D, a base line of the profile is nonuniform, while in FIG. 7E, the base line has been corrected to be uniformly zero. It can be understood therefrom that a shading component added on the image has been appropriately corrected. In addition, optical crosstalk from an adjacent well has been reduced based on a local brightness variation pattern of FIG. 7A. The appropriate correction of artifact components including the base line and the optical crosstalk improves accuracy of the negative or positive determination, thereby leading to improved analysis accuracy.

Further, Table 1 shows an example in which DNA concentrations (relative values) were actually measured through use of fluorescence analysis using beads. In a case with correction, clearly different values are exhibited for the respective DNA concentrations, and the detection limit is a DNA concentration of 0.001 or less. However, in a case without correction, almost equivalent values are exhibited for the DNA concentrations of 0 and 0.001, and the detection limit is between the DNA concentrations of 0.01 and 0.001. This result is ascribable to improvements in S/N and accuracy of the nucleic acid analysis due to removal of shading through correction.

TABLE 1

DNA concentrations (relative values)

| | 1 | 0.1 | 0.01 | 0.001 | 0 |
|---|---|---|---|---|---|
| with correction | 3.081 | 1.622 | 0.572 | 0.081 | 0.008 |
| without correction | 1.680 | 0.639 | 0.036 | 0.002 | 0.003 |

The specific nucleic acid analysis devices have been described above, but the present invention is not limited thereto.

According to the present invention, an artifact component is determined from information corresponding to a portion other than a plurality of individual independent separated compartments, and the artifact component is used to correct information corresponding to the plurality of individual separated compartments, to thereby enable the analysis of nucleic acid with artifacts removed or reduced.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-023910, filed Feb. 18, 2022, which is hereby incorporated by reference herein in its entirety.

What is claimed is:

1. A nucleic acid analysis device comprising:
a distribution unit;
a labeling unit;
an information acquisition unit; and
an information processing unit,
wherein the information processing unit includes:
an information extraction unit;
an artifact component determination unit;
a correction unit; and
an identifying unit,
wherein the distribution unit is configured to distribute a sample including nucleic acid and a reagent to a plurality of individual separated compartments to form a set including the plurality of individual separated compartments and a portion other than the plurality of individual separated compartments,
wherein the labeling unit is configured to cause a change in each of the plurality of individual separated compartments to which the sample including target nucleic acid is distributed so that the each of the plurality of individual separated compartments to which the sample including target nucleic acid is distributed and each of the plurality of individual separated compartments to which a sample excluding target nucleic acid is distributed are distinguishable from each other by the identifying unit,
wherein the information acquisition unit is configured to acquire information corresponding to the set,
wherein the information extraction unit is configured to extract, from the acquired information corresponding to the set, each of information corresponding to the plurality of individual separated compartments and information corresponding to the portion other than the plurality of individual separated compartments,
wherein the artifact component determination unit is configured to determine an artifact component from the extracted information corresponding to the portion other than the plurality of individual separated compartments,
wherein the correction unit is configured to use the artifact component to correct the extracted information corresponding to the plurality of individual separated compartments, and
wherein the identifying unit is configured to identify, based on the corrected information corresponding to the plurality of individual separated compartments, each of the plurality of individual separated compartments in which a magnitude of the change exceeds a predetermined threshold.

2. The nucleic acid analysis device according to claim 1,
wherein the change comprises generation of fluorescence, and
wherein the information corresponding to the set which is acquired by the information acquisition unit includes a fluorescent image of the set.

3. The nucleic acid analysis device according to claim 1, wherein the information corresponding to the set includes a bright-field image of the set.

4. The nucleic acid analysis device according to claim 1 wherein the information extraction unit is configured to use a default mask based on a shape of each of the plurality of individual separated compartments in the set.

5. The nucleic acid analysis device according to claim 1,
wherein the plurality of individual separated compartments comprise wells including the sample including nucleic acid and the reagent that have been distributed, and
wherein the set comprises a well plate including the wells.

6. The nucleic acid analysis device according to claim 5, wherein the portion other than the plurality of individual separated compartments includes a part of a region other than the wells of the well plate.

7. The nucleic acid analysis device according to claim 1,
wherein the plurality of individual separated compartments comprise liquid droplets including the sample including nucleic acid and the reagent that have been distributed, and
wherein the set includes the liquid droplets and a dispersion medium.

8. The nucleic acid analysis device according to claim 7, wherein the portion other than the plurality of individual separated compartments includes a part of the dispersion medium.

9. The nucleic acid analysis device according to claim 1, wherein the artifact component determination unit is configured to determine the artifact component through estimation involving approximation using a basis function.

10. The nucleic acid analysis device according to claim 9, wherein the basis function comprises a linear function.

11. The nucleic acid analysis device according to claim 1, wherein the artifact component determination unit is configured to determine the artifact component through estimation using a regularization term.

12. The nucleic acid analysis device according to claim 1, wherein the artifact component determination unit is configured to determine the artifact component through estimation involving approximation using a polynomial.

13. The nucleic acid analysis device according to claim 1, wherein the artifact component determination unit is configured to determine the artifact component through calculation using binning.

14. The nucleic acid analysis device according to claim 2,
wherein the information and the artifact component each include information on coordinates and a brightness at the coordinates, and
wherein the correction unit is configured to perform one of:
subtracting the brightness of the artifact component from the brightness of the information corresponding to the plurality of individual separated compartments; or
dividing the brightness of the information corresponding to the plurality of individual separated compartments by the brightness of the artifact component.

15. The nucleic acid analysis device according to claim 2,
wherein the reagent contains an effector protein, crRNA to be bound to the target nucleic acid, and a reporter molecule,
wherein the crRNA is bound to the target nucleic acid to activate the effector protein, and
wherein the labeling unit is configured to modify the reporter molecule by the activated effector protein to generate fluorescence.

16. A nucleic acid analysis method comprising:
a distribution step;
a labeling step;
an information acquisition step;
an information extraction step;
an artifact component determination step;
a correction step; and
an identifying step,
wherein the distribution step includes distributing a sample including nucleic acid and a reagent to a plurality of individual separated compartments to form a set including the plurality of individual separated compartments and a portion other than the plurality of individual separated compartments,
wherein the labeling step includes causing a change in each of the plurality of individual separated compartments to which the sample including target nucleic acid is distributed so that the each of the plurality of individual separated compartments to which the sample including target nucleic acid is distributed and each of the plurality of individual separated compartments to which a sample excluding target nucleic acid is distributed are distinguishable from each other in the identifying step,
wherein the information acquisition step includes acquiring information corresponding to the set,
wherein the information extraction step includes extracting, from the acquired information corresponding to the set, each of information corresponding to the plurality of individual separated compartments and information corresponding to the portion other than the plurality of individual separated compartments,
wherein the artifact component determination step includes determining an artifact component from the extracted information corresponding to the portion other than the plurality of individual separated compartments,
wherein the correction step includes using the artifact component to correct the extracted information corresponding to the plurality of individual separated compartments, and
wherein the identifying step includes identifying, based on the corrected information corresponding to the plurality of individual separated compartments, each of the plurality of individual separated compartments in which a magnitude of the change exceeds a predetermined threshold.

17. A non-transitory storage medium storing a program for causing a computer to execute the nucleic acid analysis method of claim 16.
